(19) United States
(12) Patent Application Publication
(10) Pub. No.: US 2007/0190090 A1
(43) Pub. Date: Aug. 16, 2007

(54) SIALAGOGUE BASED ORAL CARE PRODUCTS

(75) Inventor: DALE G. BROWN, Wharton, TX (US)

Correspondence Address: BANNER & WITCOFF, LTD., 28 STATE STREET, 28th FLOOR, BOSTON, MA, 02109-9601, US

(73) Assignee: WhiteHill Oral Technologies, Inc., Stafford, TX (US)

(21) Appl. No.: 11/671,075

(22) Filed: Feb. 5, 2007

Related U.S. Application Data
(60) Provisional application No. 60/771,106, filed on Feb. 7, 2006.

(51) Int. Cl. A61K 9/107; A61K 9/68

(52) U.S. Cl. 424/401; 424/49; 424/48; 424/440

(57) ABSTRACT

Biofilm-responsive oral care products containing emulsion/sialagogue mixtures, provide the user with increased and prolonged saliva flow.

### PRIORITY CLAIM

This application claims priority from commonly owned and copending U.S. Provisional Patent Application Ser. No. 60/771,106, filed 7 Feb. 2006, the disclosure of which is hereby incorporated herein by reference.

### CROSS-REFERENCE TO RELATED APPLICATIONS

This application is related to copending application Ser. No. 11/349,042, filed 7 Feb. 2006, the disclosure of which is hereby incorporated herein by reference.

### FIELD OF INVENTION

The field of the present invention relates to biofilm-responsive oral care products containing emulsion/sialagogue mixtures, which products indicate increased and prolonged saliva flow. For purposes of the present invention, oral care products include confectioneries, rinses, sprays, gels, pastes tablets containing emulsion/sialagogue mixtures, which provide the user with increased and prolonged saliva flow.

### BACKGROUND OF THE INVENTION

Oral care products used to carry out oral hygiene include: certain confectioneries, rinses, sprays, gels, pastes and tablets. All of these oral care products can be upgraded with emulsion/sialagogue mixtures to make them more biofilm-responsive and increase and prolong saliva flow.

Proper use of the various oral care products referenced above is necessary to clean those tooth surfaces which can be reached by the bristles of a toothbrush, the swishing action of rinses, etc.

Historically, the purpose of oral care products was to:
(1) dislodge and remove any decomposing food material, debris, etc., that had accumulated in the oral cavity, and
(2) dislodge and remove as much as possible the growth of bacterial material (plaque, tartar, calculus . . . eventually to be classified as biofilm) that had accumulated on tooth surfaces since the previous cleaning.

Effective oral hygiene requires that three control elements be maintained by the individual:
(1) Physical removal of stains, plaque and tartar. This is accomplished in the strongest sense by scraping and abrasion in the dentist's office during prophylaxis, scaling or root planing. Self administered physical procedures are required frequently between visits to the oral care professional and range from tooth brushing with an appropriate abrasive toothpaste through use of the various interproximal dental devices.
(2) Surfactant Cleaning. This is required to remove: food debris and staining substances before they adhere to the tooth surface; normal dead cellular (epithelial) material which is continually sloughed off from the surfaces of the oral cavity and microbial degradation products derived from all of the above. Research has shown that the primary source of bad breath is the retention and subsequent degradation of dead cellular material sloughed off continuously by the normal, healthy mouth. Besides the obvious hygienic and health benefits related to simple cleanliness provided by ore care products containing surfactants, there is an important cosmetic and sense-of-well-being benefit provided by those oral care products containing surfactants.
(3) Frequency of Cleansing. This is perhaps the most difficult to provide in today's fast-paced work and social environment. Most people recognize that their teeth should be brushed at least 3 times a day and cleaned interproximally at least once a day. The simple fact is that most of the population brush once a day, some brush morning and evening, but precious few carry toothbrush and dentifrice to use the other three or four times a day for optimal oral hygiene. Consumer research suggests that the population brushes an average of about 1.3 times a day. Most surprising, less than 15% of adults use an interdental dental device regularly.

The classification of plaque as a biofilm is considered a major advance in the development of more effective oral care products. See the following biofilm references:

Greenstein and Polson, J. Periodontol., May 1998, 69:5:507-520; van Winkelhoff, et al., J. Clin. Periodontol., 1989, 16:128-131; and Wilson, J. Med. Microbiol., 1996, 44:79-87.

Biofilms are defined as “. . . matrix-enclosed bacterial population adherent to each other and to the surface or intersurfaces. These masses secrete an exopolysaccharide matrix for protection. Considerably higher concentrations of drugs are needed to kill bacteria in biofilms than organisms in aqueous suspensions.”

Costerton, J. W., Lewandowski, Z., DeBeer, D., Caldwell, D., Korber, D., James, G. Biofilms, the customized microniche. J. Bacterio., 1994, 176:2137-2142.
The unique attributes of biofilms are being recognized as increasingly important in the 1990's. Future studies into the mode of growth of biofilms will allow manipulation of the bacterial distribution.

Douglass, C. W., Fox, C. H. Cross-sectional studies in periodontal disease: Current status and implications for dental practice. Adv. Dent. Res., 1993, 7:26-31.

Greenstein, G. J., Periodontal response to mechanical non-surgical therapy: A review. Periodontol., 1992, 63:118-130.
Mechanical therapy remains effective with caveats to compliance and skill of therapists.

Marsh, P. D., Bradshaw, D. J. Physiological approaches to the control of oral biofilms. Adv. Dent. Res., 1997, 11:176-185.
Most laboratory and clinical findings support the concept of physiological control. Further studies will reveal details of biofilm diversity.

Page, R. C., Offenbacher, S., Shroeder, H., Seymour, G. J., Komman, K. S., Advances in the pathogenesis of periodontitis: Summary of developments, clinical implications and future directions. Periodont. 2000, 1997, 14:216-248.
Genetic susceptibility to three oral anaerobic bacteria plays an important part in the progression of periodontitis. Acquired and environmental risk factors exacerbate the problem. Mechanical disruption will remain an effective and essential part of periodontal therapy.

Papapanou, P. N., Engebretson, S. P., Lamster, I. B. Current and future approaches for diagnosis of periodontal disease. NY State Dent. J., 1999, 32-39.
New techniques are available such as a novel pocket depth measurement device, microscopic techniques, immunoassay, DNA probes, BANA hydrolysis tests. These more clearly define the nature of periodontitis.

Slavkin H. H. Biofilms, microbial ecology and Antoni Van Leeuwenhoek. J. Am. Dent. Assoc., 1997, 128:492-495.

DuPont, G. A. Understanding dental plaque; biofilm dynamics. J. Vet. Dent., 1977, 14:91-94.

Marsh P. D., Bradshaw, D. J. Dental plaque as a biofilm. J. Industrial Microbiology, 1995, 15:169-175.

Shearer, B. J. Biofilm and the dental office. J. Am. Dent. Assoc., 1996, 127:181-189.

Meyer, K. F. The present status of dental bacteriology. J. Am. Dent. Assoc., 1917, 4:966-996.

Haffajee, A. D., Socransky, S. S. Evidence of bacterial etiology: a historical perspective. Periodontology 2000, 1994, 5:7-25.

Willmann, D. E., Chaves, E. S. The role of dental plaque in the etiology and progress of inflammatory periodontal disease. In Harris N O, Garcia-Godoy F eds. Primary preventive dentistry. Stamford, Conn.: Appleton & Lange, 1999, 63-76.

Kimball, G. D. The relationship of materia alba and dental plaque to periodontal disease. J. Periodontol. 1952, 23:16-169.

Keyes, P. H., Jordan, H. V. Periodontal lesions in the Syrian hamster. III. Findings related to an infectious and transmissible component. Arch. Oral Biol., 1964, 9:377-400.

Listgarten, M. A. Electron microscopic observations of the bacteria flora of acute necrotizing ulcerative gingivitis. J. Periodontol., 1965, 36:328-339.

Haffajee, A. D., Socransky, S. S. Microbial etiological agents of destructive periodontal diseases. Periodontol. 2000, 1994, 5:78-111.

Consensus report on periodontal diseases: pathogenesis and microbial factors. Ann. Periodontol., 1996, 1:12-32.

Wilson, M., Gibson, M., Strahan, D., et al. A preliminary evaluation of the use of a redox agents in the treatment of chronic periodontitis. J. Periodont. Res., 1992, 27:522-527.

Adapted from Marsh and Bradshaw. Physiological approaches to the control of oral biofilms. Adv. Dent. Res., 1997, 11:176-185.

The classification of plaque as a biofilm calls for more effective oral care products, with respect to removing, disrupting and/or controlling biofilms, increasing and prolonging saliva flow.

Xerostomia, the subjective feeling of oral dryness, is primarily caused by a marked decrease in the function of the salivary glands. Although not a disease, it may herald the onset, or signal the presence of a number of serious systemic diseases and conditions. Among these are the intake of xerogenic drugs, autoimmune diseases and radiation to the head and neck. Moreover, it may profoundly affect the soft and hard tissues of the mouth and interfere with alimentation and speech.

The dental ramifications of salivary gland hypofunction are quite well known. Saliva is a major protector of the tissues and organs of the oral cavity. In its absence, both the hard and soft tissues of the mouth may be severely damaged; the development of caries increases, the oral mucosa may become infected and/or ulcerated, and functions connected with the intake and digestion of foods may be impaired.

From an evolutionary viewpoint, the oldest function of the salivary glands has been to supply lubricatory molecules, not only to coat the food but also the soft and hard tissues. The lubricatory film allows food to travel easily through the digestive system, and provides smooth tissue surfaces with minimal function. Without appropriate lubrication, food is retained and impacted around teeth, making eating difficult and unpleasant and increasing plaque formation.

### SUMMARY OF THE INVENTION

The present invention discloses and claims various oral care products containing a surfactant and a sialagogue, or a saliva soluble emulsion/sialagogue mixture and associated methods for:
(a) controlling, and/or disrupting, and/or physically removing biofilms, while
(b) increasing and prolonging saliva flow.
   Preferably, the emulsion/sialagogue mixture comprises:
(1) a substantially water-free emulsion having a surfactant continuous phase and a coating substance, and
(2) at least one sialagogue.

The present invention also discloses and claims various biofilm-responsive oral care products comprising substantially water-free emulsions containing a sialagogue, which are suitable for increasing and prolonging saliva flow.

### PREFERRED EMBODIMENTS

One embodiment of the present invention further discloses and claims various treatments for xerostomia including impaired salivary function in patients with noninsulin-dependent diabetes mellitus.

Accordingly, one embodiment of the present invention comprises biofilm-responsive oral care products suitable for controlling, disrupting and removing biofilms, while increasing and prolonging saliva flow.

A further embodiment of the present invention comprises saliva soluble oral care products containing a releasable emulsion/sialagogue, thereby rendering the oral care product biofilm-responsive and suitable for increasing and prolonging saliva flow.

Another embodiment of the invention comprises a self-treatment means comprising an oral care product suitable for routinely removing, controlling and disrupting biofilms formed on tooth surfaces, and for coating residual biofilms that remain after use of the oral care product with emulsion/sialagogue, while also increasing, and prolonging saliva flow, thereby maintaining control of biofilms.

Yet another embodiment of the invention comprises a patient self-treatment method for periodically (i.e., hourly, daily, weekly, monthly—as needed) removing and disrupting biofilms that form on tooth surfaces, and treating residual biofilms with an emulsion/sialagogue or a sialagogue coating that helps control biofilms, while simultaneously increasing, prolonging saliva flow.

Still another embodiment of the invention comprises oral care products and associated methods for: (a) removing, disrupting and controlling the supragingival microbiological burden associated with biofilms, and (b) increasing and prolonging saliva flow, in at-risk adults suffering from dry mouth.

The present invention additionally discloses and claims various methods for manufacturing various oral care products treated with emulsion coatings containing one or more sialagogues.

### DEFINITIONS

For purposes of describing the present invention, the following terms are defined as set out below:

As used herein, the term “sialagogue(s)” is defined as a natural or synthetic compound or mixture of compounds that cause an increase in saliva in the mouth. In other words, sialagogues are substances that stimulate the production of saliva.

There are two important types of sialagogues:

(A) Gustatory sialagogues, i.e., materials related to the sense of taste, such as particular foods and flavors. Particularly preferred sialagogues include: ascorbic acid, black pepper, ginger, licorice, pilocarpine, affinin, spilanthol, bethanechol chloride, cayenne pepper, echinacea, verba santa, bay berry, sanguinarine, ginseng, kava, kudzu, capsaicin, zingerone, eugenol, and piperine.

(B) Pharmaceutical sialagogues (also called parasympathomimetic agents) which improve salivation.

Examples of other preferred sialagogues of both types, useful in the present invention, are the following:
1. Plant extracts including: amides of vegetable origin including:
   (a) affinin, i.e., N-isobutyl-2,6,8-decatrienamide,
   (b) Ciluan Root derivatives, including Heliopsis longipes,
   (c) bioactive N-isobutylamides from buds of Spilanthes acmella,
   (d) alkamides present in flavoring plants including affinin and capsaicin, and
   (e) N-alkyl-carboxamide compounds, including 3-(1-menthoxy)propane-1,2 diol1(2-hydroxyphenyl)-4-(3-nitrophenyl)-1,2,3,5-tetrahydropyrimidine-2-one; and
2. compositions such as described in U.S. Pat. Nos. 5,585,424; 6,780,443; 6,890,567; 6,899,901; U.S. Patent Publication No. 2003/0215532 and U.S. Patent Publication No. 2004/0052735. See also: Journal of the Society of Cosmetic Chemists, 29:185-200 (1988) H. R. Watson. Preferably such compositions contain at least one Jambu Oleoresin and one Spilanthol;
3. “Spilanthes” which are a strong anti-bacterial herb with in-vitro activity against such common pathogens as: Escherichia coli, Klebsiella pneumoniae, Proteus vulgaris, Pseudomonas aeruginosa, Salmonella gallinarum and Staphylococcus albus. Spilanthes also inhibits the yeast/fungal organism Candida albicans, which is responsible for the nearly epidemic condition known as candidiasis (thrush);
4. “Heliopsis longipes”, which are herbaceous plant species found in Mexico, have long been used there primarily as a spice or flavoring, as chewing the root causes numbness and tingling in the mouth and tongue and stimulates salivation. Little, Jr., E. L., Journal of the Washington Academy of Sciences, Vol. 38, No. 8, pp. 269-274 (Aug. 15, 1948). These roots were also chewed to relieve toothache. Id.
5. Extracts from the roots of Heliopsis longipes have been used in a few medical applications. Id. For example, reportedly, such an extract has been used for treating colds and pneumonia, and an alcoholic extract has been tested for use as an anesthetic for tooth extraction. Id. It has also been reported that an extract of these roots possesses antiseptic properties. Molina-Torres, J., et al. Antimicrobial properties of alkanides present in flavoring plants traditionally used in Mesoamerica: affinin and capsaicin, Journal of Ethnopharmacology, Vol. 64, Iss. 3, pp. 241-248 (March 1999). A crude methanol extract of Heliopsis longipes roots has been described as having the potential to generate anti-infective agents, although this extract reportedly does not show any activity in plate diffusion tests against either E. coli (Gram negative bacteria) or B. subtilis (Gram positive bacteria). Id.; and Gutierrez-Lugo, M. T., et al., Antimicrobial and cytotoxic activities of some crude drug extracts from Mexican Medicinal plants, phyomedicine, Vol. 2 (4), pp. 341-347 (1996). An ethanol extract of Heliopsis longipes roots has been reported as having variable bactericidal effects on E. coli and S. aureus, Romero-R., C. M., et al., Preliminary Studies of the Antibacterial, Insecticidal, and Toxicological Effects of Chiluan Root (Heliopsis longipes), as translated, Veterinaria Mexico, pp. 151-156 (1989).
6. Heliopsis longipes roots are known to contain a bioactive alkamide, affinin, identified as N-isobutyl-2E,6Z,8E-decatrienamide or N-isobutyldeca-trans-2, cis-6, -trans-8-trienamide. Respectively, Id.; and Crombie, L., et al., Amides of Vegetable Origin, part X. The Stereochemistry and Synthesis of Affinin, Journal of Chemical Society, pp. 4970-4976 (1963).
7. Affinin has also been identified as N-isobutyl2,6,8-decatrienoamide in one publication, in another publication, and N-isobutyldodeca-2-trans-6-cis-8-trans-trienamide in another publication. Respectively, Jacobson, M., et al., Correction of the Source of “Affinin” (N-Isobutyl-2,6,8-Decatrienoamide, Journal of Organic Chemistry 12, pp. 731-732 (1947); and Ogura, M., et al., Ethnopharmacologic studies. I. Rapid solution to a problem-oral use of Heliopsis longipes-by means of multidisciplinary approach, Journal of Ethnopharmacology, 5, pp. 215-219 (1982) (emphasis added). Purified affinin, prepared from an ethanol extract of Heliopsis longipes roots, has been reported as being toxic to certain microorganisms, the toxicity varying for Gram positive and Gram negative bacteria. Molina-Torres, J., et al.
8. An aqueous solution of affinin, prepared from a powder of an ethanol extract of Heliopsis longipes roots, has also been reported as having an analgesic effect when administered orally to mice at doses from 2.5 to 10.0 mg/kg, with severe depression of normal motor activity and two out of five deaths occurring at the highest dose. Ogura, M., et al. In the one publication where affinin is identified as N-isobutyl-dodeca-2-trans-6-cis-8-trans-trienamide, it was said to be identical with spilanthol, the pungent principle of several Spilanthes species. Ogura, M., et al. However, in the publication of Little, Jr., affinin is said to be similar to spilanthol, which has been isolated from flower heads of a species of Spilanthes, Little, Jr., E. L., at p. 270. The flowers and leaves of Spilanthes acmella L. var. oleracea Clarke are reported as having been used as a spice and as a folk medicine for stammering, toothache, stomatitis and throat complaints. Ramsewak, R. S. et al., Bioactive N-isobutylamides from the flower buds of Spilanthes acmella, Phytochemistry 51, pp. 729-732 (1999).
   The Heliopsis longipes extract may be prepared using standard means or methods, such as by contacting the plant material with an appropriate solvent to prepare a botanical tincture, or by any other conventional means or method, such as by CO₂ extraction, freeze-drying, spray-drying, and the like. (See Gennaro A R; Remington: The Science and Practice of Pharmacy, Mack Publishing Company, Easton Pa. 1995 and The United States Pharmacopeia 22 Rev, and The National Formulary (NF) 17 ed, USP Convention, Rockville, Md., 1990.) The extract is prepared using a root or roots of Heliopsis longipes and a solvent, such as water combined with other solvents, an organic solvent, such as hexane and glycerin, or an alcohol, such as ethanol, or any combination thereof. Preferably, an alcohol or a hydro-alcohol solvent is used, and most preferably, ethanol or a combination of ethanol and water is used.
   The resulting extract is typically composed of a wet or liquid component that is light brown to golden in color and a dry or solid component, in amounts of about 90.0 to about 99.9 weight percent, such as about 98 weight percent, and about 10 to about 0.01 weight percent, such as about 2 weight percent, respectively, relative to the extract. The composition, including the extract in the wet-dry form just described, may be formulated as a powder or paste, such as a powder including about 66.6 weight percent extract and 33.4 weight percent carrier on a wet basis; or about 0.01 to about 100 weight percent extract on a dry basis-including the natural product sprayed on itself, such as about 2 to 10 weight percent extract on a dry basis, or in any combination or permutation for either method—wet or dry.
   All of the foregoing references regarding sialagogues are incorporated herein by reference.

“Oral care products of the invention” are defined as confectioneries, sprays, rinses, gels, pastes, tablets, etc., such as described and claimed in the following U.S. patents:

| | | | | |
|---|---|---|---|---|
| 6,723,305; | 6,500,409; | 6,464,963; | 6,379,652; | 6,375,933; |
| 6,365,560; | 6,306,371; | 6,290,935; | 6,258,343; | 6,254,857; |
| 6,235,268; | 6,214,320; | 6,174,516; | 6,136,298; | 6,129,907; |
| 6,110,445; | 6,096,293; | 5,998,487; | 5,989,526; | 5,976,507; |
| 5,958,381; | 5,932,192; | 5,912,274; | 5,560,905; | 5,009,882; |
| 4,959,204; | 6,927,053; | 6,770,266; | 6,723,305; | 6,692,726; |
| 6,669,930; | 6,669,929; | 6,592,849; | 6,500,409; | 6,464,963; |
| 6,451,291; | 6,447,758; | 6,419,903; | 6,416,744; | 6,379,654; |
| 6,379,652; | 6,375,933; | 6,346,235; | 6,333,024; | 6,315,986; |
| 6,306,371; | 6,290,935; | 6,290,933; | 6,254,857; | 6,235,268; |
| 6,214,320; | 6,207,138; | 6,180,089; | 6,159,459; | 6,149,894; |
| 6,136,298; | 6,129,907; | 6,126,923; | 6,113,884; | 6,110,446; |
| 6,110,445; | 6,106,812; | 6,096,293; | 5,998,487; | 5,989,926; |
| 5,976,508; | 5,976,507; | 5,968,480; | 5,958,381; | 5,912,274; |
| 5,876,701; | 5,885,871; | 5,853,704; | 5,851,514; | 5,851,513; |
| 5,849,267; | 5,843,409; | 5,843,406; | 5,840,281; | 5,833,956; |
| 5,814,304; | 5,788,951; | 5,780,015; | 5,776,435; | 5,766,574; |
| 5,756,073; | 5,730,959; | 5,728,756; | 5,723,500; | 5,723,107; |
| 5,723,105; | 5,698,182; | 5,693,314; | 5,690,913; | 5,690,911; |
| 5,686,064; | 5,683,680; | 5,681,548; | 5,630,999; | 5,820,852; |
| 5,603,920; | 5,560,905; | 5,281,410; | 5,145,666; | 5,011,830; |
| 5,009,882; | 4,965,067; | 4,986,981; | and 4,959,204 | |

All of the foregoing are incorporated herein by reference.

“Dry mouth, at-risk patients” defines those patients suffering from reduced saliva flow, such as those who have one or more chronic diseases which they regularly treat with medicine having an adverse side reaction that causes dry mouth and/or patients who receive treatments that adversely affect saliva flow such as radiation therapy and chemotherapy that typically cause dry mouth. See also:

(a) McDonald E. and Marino C. “Dry Mouth: Diagnosing and treating its multiple causes.” Geriatrics 1991; 46 (Mar. 61-63);

(b) Sreebyn L. M. and Valdini A. “Xerostomia Part 1: Relationship to other oral symptoms and salivary gland hypofunction.” Oral Surg. Oral Med. Oral Pathol. 1988, 66:451-8;

(c) Sreebyn L. M. “Salivary flow in health and disease.” Compend. Contin. Educ. Dent. Suppl. No. 13:461-469;

(d) Mandel I. W. “The role of saliva in maintaining oral homeostasis.” JADA Vol. 119, August 1989; 298-304; and

(e) Mandel I. W. “The functions of saliva.” J. Dent. Res. 66 (Spec Iss) 623-627; February 1987.

All of the foregoing are incorporated herein by reference.

“Saliva flow” is defined as an important element in oral hygiene. Saliva flow tends to wash the mouth of food and contaminants, promotes a balanced ecology of the oral cavity including the gums and teeth, and refreshes the mouth. Stimulating saliva flow alleviates xerostomia and the sensations and perception of dry mouth; reduces oral bacteria, dental caries, halitosis, gingivitis, periodontitis, and oral plaque (biofilms); and promotes the healing of and alleviation of oral lesions, such as lesions present in the mouth, including lesions induced by stomatitis, herpes, and the like.

“Emulsions” are defined as surfactant coating combinations where the surfactant is the continuous phase and the coating substance is the discontinuous phase and indicates:
(a) those emulsions described in the following U.S. Pat. Nos. 4,950,479; 5,032,387; 5,538,667; 5,561,959; and 5,665,374, which are hereby incorporated by reference,
(b) various emulsions, such as described in U.S. Pat. Nos. 5,908,039; 6,080,495; 4,029;113; 2,667,443; 3,943,949; 6,026,829; 5,967,155 and 5,967,153, which are hereby incorporated by reference, and
(c) those substantive saliva soluble emulsions described and claimed in U.S. Pat. Nos. 6,907,889; 6,609,527; 6,916,880 and 6,545,077, which are hereby incorporated by reference.

All of the foregoing emulsions can contain biofilm-responsive levels of one or more substances suitable for controlling and disrupting biofilms and at least one sialagogue for prolonging and increasing saliva flow.

As used herein, the phrase “wax emulsions” are comprised of a suitable wax, such as microcrystalline wax, paraffin wax, beeswax, and the like, emulsified into a suitable emulsifier or surfactant such as ceteareth-20 or polysorbates 60, with or without the addition of long chain alcohols such as stearyl alcohols, into which is admixed small amounts of flavors, sialagogues, sweeteners, and other low-level active ingredients commonly used in oral care products.

As used herein, the terms “MICRODENT®” and “ULTRAMULSION®” refer to emulsions of polydimethylsiloxane at various molecular weights in various poloxamer surfactants as described and claimed in U.S. Pat. Nos. 4,911,927; 4,950,479; 5,032,387; 5,098,711; 5,165,913; 5,538,667; 5,645,841; 5,651,959 and 5,665,374. It has been discovered that sialagogues can readily be added to these emulsions as set forth in greater detail herein.

As used herein, the term “inverse wax emulsion” defines an emulsion wherein the continuous phase is a suitable wax, such as microcrystalline wax, paraffin wax, beeswax, and the like, and the discontinuous phase is a surfactant. For example, emulsified as the discontinuous phase is a saliva-soluble surfactant solution of flavors, sweeteners, low-level active ingredients and other modifiers, along with sialagogues, which are released into the oral cavity in lesser amounts than the previously described “saliva-soluble, crystal-free coatings”.

As used herein, the term “antimicrobial” includes various active ingredients that: control, disrupt and/or kill various microbiota associated with residual biofilms, which remain on tooth surfaces after treatment with the oral care products of the present invention. These include topical antimicrobials, such as: chlorhexidine digluconate (chlorhexidine), triclosan, benzylalkonium chloride, cetylpyridinium chloride, iodine, metronidazole and microbially active essential oils, such as thymol, menthol, etc.

A. Specifically, first generation antimicrobials include:
1. quaternary ammonium compounds such as benzethonium chloride, cetylpyridinium chloride,
2. phenolic compounds such as thymol and eucalyptol in a mixture of methyl salicylate, benzoic acid and boric acid and phenol,
3. natural extracts (flavor oils) known to possess antimicrobial properties, and
4. sanguinarine extract, alone or in combination with zinc chloride, or zinc chloride alone.

B. Second generation antimicrobials include: antibacterial agents with substantivity such as chlorhexidine, either free base or as the gluconate or other suitable salts, including alexidine, octenidine and stannous fluoride.

As used herein, the term “biofilm-responsive” is defined as the property of emulsions containing at least one sialagogue to work cooperatively with oral care products to physically remove, disrupt and/or control biofilms and the microbiological burden associated with biofilms and to treat residual biofilms with sialagogues, while increasing and prolonging saliva flow.

As used herein, the term “flavorants” are defined as flavoring components suitable for emulsifying in saliva soluble coatings exemplified by the following substances: menthol, anise oil, benzaldehyde, bitter almond oil, camphor, cedar leaf oil, cinnamic aldehyde, cinnamon oil, citronella oil, clove oil, eucalyptol, heliotropine, lavender oil, mustard oil, peppermint oil, phenyl salicylate, pine oil, pine needle oil, rosemary oil, sassafras oil, spearmint oil, thyme oil, thymol, wintergreen oil, lemon and orange oils, vanillin, spice extracts and other flavoring oils generally regarded as safe (GRAS) by health authorities.

As used herein, the term “additional adjuvants” refers to additional ingredients that can be added to the emulsion/sialagogue mixture to provide color, or sweetening effects, as desired. Examples of suitable sweetening agents include sorbitol, sodium cyclamate, saccharine, commercial materials such as Nutrasweet® brand of aspartame and xylitol. Citric acid or acetic acid is often utilized as a flavor modifier and is generally used in amounts of about 1.0 to about 20 percent by weight, preferably about 2.0 percent to about 15 percent by weight.

As used herein, the term “buffering ingredient” refers to substances that may also be added to the flavored compositions of the invention in order to prevent natural degradation of the flavoring components or therapeutically active ingredients. Generally, the pH of these compositions is adjusted from about 3.5 to about 8, depending on the chemistry of the active ingredient most requiring protection. Buffering ingredients such as an alkali metal salt of a weak organic acid, for instance, sodium benzoate, sodium citrate, sodium phosphate, sodium bicarbonate or potassium tartrate is generally added in an amount of about 0.1 to about 1.0 percent by weight. Other buffering agents such as weak organic acids or salts of weak bases and strong acids such as boric acid, citric acid, ammonium chloride, etc., can also be used in similar concentrations.

As used herein, the term “stabilizers” refers to substances that are often added along with the flavorant to the oral care products for additional control, such as:
(a) sodium benzoate, sodium or potassium sorbate, methyl paraben, propylparaben and others approved for ingestion, and
(b) chemical oxidative control substances, such as ethylene-diaminetetraacetic acid, BHA, BHT, propyl gallate and similar substances approved for ingestion. Concentration levels of these stabilizers comply with industry and regulatory standards.

As used herein, the term “cleaners” refers to essentially all surfactants suitable for use in the oral cavity and suitable for the oral care products of the present invention.

As used herein, the phrase “chemotherapeutic ingredients” refers to those substances other than sialagogues suitable for addition to the oral care products of the present invention that impart therapeutic effects to the oral cavity including antimicrobials; anti-tartar and anti-plaque substances; remineralizing, desensitizing, NSAID and antibiotic ingredients, and the like. Specific chemotherapeutic ingredients suitable for the present invention include: stannous fluoride, potassium nitrate, cetylpyridinium chloride (CPC), triclosan, metronidazole, chlorhexidine, aspirin and doxycycline.

As used herein, the term “viscosity control agents” includes those substances generally known in the food and consumer products, but not commonly used in oral care products, which are selected from natural and synthetic gums such as: carrageenan, gum tragacanth, methyl celluloses and derivatives there of such as hydroxymethyl methyl cellulose, polyvinyl pyrrolidone, and hydrophilic carboxyvinyl polymers such as those sold under the trademark Carbopol 934. Generally, about 0.01 percent to about 10 percent of one or more viscosity control agents is used. Often these substances are used as dry powders directly incorporated as a third phase into the melt-emulsion mixture. With appropriate control of the active water content, some or all of these dry viscosity agents could be substituted with pre-gelled viscosifiers containing no free water.

### DETAILED DESCRIPTION OF THE INVENTION

There are five oral care, biofilm-responsive factors that can be delivered by the oral care products of the invention. These include abrasion, removal of precursors to plaque, altering the attachment of plaque to a surface, enhancing saliva flow, and anti-inflammatory activity:
1. Abrasive removal of the plaque film, once it has firmly adhered to the tooth surface, is the only totally effective cleansing mechanism. Again, professional dental hygiene is most effective, but recently, a number of special abrasive toothpastes have been accepted by dental organizations for partially removing supragingival adhered plaque and the tartar which subsequently forms from the plaque;
2. Removal of plaque precursors requires the reduction of food sources and building blocks required for the bacteria to synthesize the mucopoly-saccharides which polymerize into the plaque film. Going far back into the chain of events leading to plaque formation and interrupting the chain has much to commend it as a sound oral hygiene strategy. However, for this technique to be effective, the plaque building blocks must be interrupted periodically (i.e., at least daily) throughout the mouth, especially at the site of plaque buildup and if possible just below the tooth-gum interface and interproximally. For reference see L. Menaker, “The Biological Basis of Dental Caries,” Chapters 5, 11, 12, 14, 16 and 18, Harper and Row (1980);
3. As to altering attachment of plaque, it has now been found that the emulsion/sialagogue mixtures described below can be incorporated into oral care products of the present invention. The release of these substances from oral care products is particularly effective in disrupting, for prolonged periods, the plaque matrix on tooth surfaces;
4. Enhancing saliva flow by increasing and prolonging saliva flow through the release of sialagogues into the oral cavity; and
5. Anti-inflammatory activity associated with sialagogues, including inhibiting pro-inflammatory cytokines and free radicals and targeting prostaglandins, which are involved in late phase of acute inflammation and pain perception.

In addition, there are three unexpected sensory-responsive factors delivered by the oral care products of the present invention, as detailed below:
1. Increase of instantaneous, during-use pleasure which increases the oral care products use frequency, duration of use and the encouragement of effective use techniques. This includes the introduction of pleasant flavors, mouthfeel and salivation which typically accompanies the consumption of pleasant foods, treats and confections. Oral care products with sialagogues combined with surfactants, emulsions or MICRODENT®, stimulate the non-olfactory (smell or aroma), non-flavor (i.e., bitter, sweet, sour and salty) sensory receptors. This produces a surprising increase of during-use pleasure of oral care products far beyond that typically expected from typical flavors or cooling ingredients such as menthol.
2. Increase of post-use pleasure to re-enforce the desire to use oral care products regularly. Oral care products with sialagogues are surprising in their longevity of effect. The reminder that “I did something good for myself” over 10 to 15 minutes after use is a surprisingly strong reinforcement of the psychological conditioning that produces good oral care habits. This is especially true since the sensory-response is throughout the oral cavity.
3. Reduction of pain without the excessive numbing of traditional mouth-pain ingredients like benzocaine and lidocaine traditionally used in various gels. Benzocaine and lidocaine create an unpleasant numbing perception while sialagogues have a simultaneous tingling sensation that actually makes the affected areas feel more “alive” even while reducing the perception of normal pain generation associated with use of interproximal devices. This surprising effect is particularly observed when the sialagogues are introduced directly to the gingival surfaces at the same time the pressure induced pain is generated.

Biofilm-responsive oral care products suitable for coating tooth surfaces with an emulsion containing at least one sialagogue include: confectioneries, rinses, sprays, gels, pastes and tablets as described in the various patents cited above.

Regular use of various oral care products of the present invention provides a unique combination of: mechanical action, detersive action, surface modification and chemotherapy with increased and prolonged saliva flow attributed to the sialagogues, resulting in:
(a) disruption of supragingival microflora, and
(a) removal of supragingival plaque (biofilm).

Supragingival chemotherapeutic disruption of microflora is achieved by the unique combination of:
(a) chemical cleansing with surfactants released from the oral care products of the present invention,
(a) prolonged modification of the surface chemistry of the microflora by the emulsion materials released, and
(b) alteration of microflora with various active ingredients contained in the oral care products.

Supragingival mechanical disruption of microflora is achieved by the unique combination of:
(a) physical disruption by the various oral care products,
(b) abrasive, disruption with abrasives released from the oral care products, and
(c) surfactant disruption resulting from the release of surfactants during use.

Chemotherapeutic removal of supragingival plaque (biofilm) is achieved by the unique combination of:
(a) chemical cleansing with surfactants released from the oral care products,
(b) modification of the surface chemistry of the plaque with emulsions, and
(c) alteration of the plaque with various active ingredients contained in the oral care products and released during use including: tetrasodium pyrophosphate, tetrapotassium pyrophosphate, etc.

Mechanical removal of supragingival plaque is achieved by the unique combination of:
(a) physical removal by certain oral care products such as pastes,
(b) abrasive removal by the abrasives released from certain oral care products including: silica, dicalcium phosphate, pyrophosphates, etc., and
(c) cleansing resulting from the release of surfactants during use.

### Emulsions Suitable for the Invention

The various oral care products of the present invention can contain sialagogues with a broad range of emulsions, such as:
1. The high melt viscosity mixtures and emulsions,
2. The medium melt viscosity mixtures and emulsions, and
3. The low melt viscosity mixtures and emulsions.

The improved oral care products of the present invention contain emulsion/sialagogue mixtures that: (a) comprise from between about 1 and about 50% by weight of oral care products, and (b) are preferably saliva soluble.

In a preferred embodiment, these emulsions contain in addition to the sialagogue, other ingredients such as: (a) antimicrobials, (b) SOFT ABRASIVES® that help physically remove biofilm (plaque) from supragingival surfaces, (c) other chemotherapeutic ingredients affecting oral health, (d) cleaners that introduce detersive effects into the areas worked by the various devices, and (e) mouth conditioners and flavorants.

Suitable liquid surfactants for the emulsions of the present invention include polyoxyethylene glycol sorbitan mono- and di-aliphatic esters represented by the general formula:

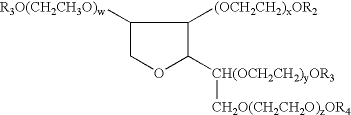

wherein R₁, R₂, R₃, R₄ and H or aliphatic acyl groups having from between about 10 and 30 carbon atoms, and the sum of w, x, y, and z is from between about 20 and about 80. These liquid surfactants are available under the trade name Emsorb®, Span®, Tween® from Cognis, N.A. and ICI. Specific examples of these include:

PEG 20 sorbitan monooleate (Tween® 80, ICI); PEG 40 sorbitan monostearate (SPAN 60 ICI) and PEG 40 sorbitan diisostearate (Eumulgin® SDI 40, Cognis N.A.).

Suitable solid surfactants include:
sodium lauryl sulfate,
sodium lauryl sarcosinate,
polyethylene glycol stearate,
polyethylene glycol monostearate,
coconut monoglyceride sulfonates,
sodium alkyl sulfate,
sodium alkyl sulfoacetates,
block copolymers of polyoxyethylene and polyoxybutylene,
allylpolyglycol ether carboxylates,
polyethylene derivatives of sorbitan esters,
propoxylated cetyl alcohol,
block copolymers comprising a cogeneric mixtures of conjugated polyoxypropylene, and polyoxyethylene compound having as a hydrophobe a polyoxypropylene polymer of at least 1200 molecular weight (these surfactants are generally described as poloxamers; specific examples are described in the Examples below) as Poloxamer 407 and Poloxamer 388,
soap powder, and
mixtures thereof.

Various chemotherapeutic agents suitable for inclusion in the emulsions of the oral care products of the present invention include:
1. anti-plaque and/or anti-tartar substances such as MICRODENT®, ULTRAMULSION®, tetrasodium pyrophosphate (TSPP);
2. tetrapotassium pyrophosphate, sodium hexametaphosphate, etc.;
3. first generation anti-biofilm agents which are antibacterial agents with limited substantivity such as oxygenating compounds, quaternary ammonium compounds, phenolic compounds and plant alkaloids, including:
   a. quaternary ammonium compounds such as benzethonium chloride and cetylpyridinium chloride (CPC),
   b. phenolic compounds such as thymol and phenol, methyl salicylate and other compositions such as benzoic acid and boric acid,
   c. natural extracts (flavor oils) known to possess antimicrobial properties including eucalyptol,
   d. sanguinaria extract, alone or in combination with zinc chloride, or zinc chloride alone,
   e. triclosan, and
   f. iodine;
4. second generation agents which are antibacterial agents with substantivity such as chlorhexidine, either free base or as the gluconate or other suitable salt, alexidine, octenidine and stannous fluoride. The treatment of the oral cavity with stannous fluoride or chlorhexidine antimicrobial containing HDPE dental devices are preferred embodiments of the present invention;
5. desensitizing agents, antibiotics, anti-thrush agents, substances to neutralize Candida sp. yeasts, anti-caries agents, antimicrobials, COX-2 agents, etc.;
6. dry mouth relieving agents;
7. NSAIDs including aspirin;
8. antibiotics including doxycycline, tetracycline and minocycline; and
9. metronidazole.
   Certain petroleum waxes are suitable and preferred additional additives for the emulsions of the present invention. These include any of a range of relatively high molecular weight hydrocarbons (approximately C₁₆ to C₅₀), solid at room temperature, derived from the higher-boiling petroleum fractions. There are three basic categories of petroleum wax: paraffin (crystalline), microcrystalline, and petroleum. Paraffin waxes are produced from the lighter lube oil distillates, generally by chilling the oil and filtering the crystallized wax they have a melting point range between 48° C. (118° F.) and 71° C. (160° F.). Fully refined paraffin waxes are dry, hard, and capable of imparting good gloss. Microcrystalline waxes are produced from heavier lube distillates arid residue (one bottoms) usually by a combination of solvent dilution and chilling. They differ from paraffin waxes in having poorly defined crystalline structure, darker color, higher viscosity, and higher melting points-ranging from 63° C. (148° F.) to 93° C. (200° F.). The microcrystalline grades also vary much more widely than paraffins in their physical characteristics: some are ductile and others are brittle or crumble easily.

Petrolatum is derived from heavy residual lube stock by propane dilution and filtering or centrifuging. It is microcrystalline in character and semi-solid at room temperature. There are also heavier grades for industrial applications, such as corrosion preventives, carbon paper, and butcher's wrap. Traditionally, the terms slack wax, scale wax and refined wax were used to indicate limitations on oil content. Today, these classifications are less exact in their meanings, especially in the distinction between slack wax and scale wax. Natural waxes such as beeswax and carnauba wax are also suitable and may provide specifically desired properties.

Examples of saliva-soluble formula modifiers include so-called water soluble waxes, such as:
liquid polyethylene glycols,
solid polyethylene glycols,
liquid polyethylene glycols,
solid polypropylene glycols, and
triacetin.

Examples of low-melt temperature, water-soluble polymers, include:
hydroxyethylcellulose,
hydroxypropylcellulose,
carboxy derivatives of cellulose, and
orally suitable saliva getting or water-soluble copolymers of various resins.

Suitable emulsions especially include those described and claimed in the various MICRODENT® and ULTRAMULSION® U.S. patents including U.S. Pat. Nos. 4,911,927; 4,950,479; 5,032,387; 5,098,711; 5,165,713; 5,538,667; 5,645,841; 5,561,959 and 5,665,374, all of which are hereby incorporated by reference.

Examples of suitable surfactants include:
sodium lauryl sulfate,
sodium lauryl sarcosinate,
polyethylene glycol stearate,
polyethylene glycol monostearate,
coconut monoglyceride sulfonates,
sodium alkyl sulfate,
sodium alkyl sulfoacetates,
block copolymers of polyoxyethylene and polyoxybutylene,
allylpolyglycol ether carboxylates,
polyethylene derivatives of sorbitan esters,
propoxylated cetyl alcohol,
block copolymers comprising a cogeneric mixtures of conjugated polyoxypropylene, and
polyoxyethylene compound having as a hydrophobe a polyoxypropylene polymer of at least 1200 molecular weight (these surfactants are generally described as poloxamers; specific examples are described in the Examples below) as Poloxamer 407 and Poloxamer 388,
soap powder, and
mixtures thereof.
Examples of suitable coating substances for the emulsions of the present invention include waxes (both natural and synthetic), such as: microcrystalline waxes, paraffin wax, carnauba, beeswax and other natural waxes, animal and vegetable fats and oils, and low-melt point, orally suitable polymers and copolymers; silicones; silicone glycol copolymers and polydimethylsiloxanes at molecular weights from between about 700 centistokes (cs) and ten million cs. (Specific examples are described in the Examples below including PDMS 2.5 million cs and ULTRAMULSION® 10-2.5.)

Sialagogue emulsions described in Formulas A-D set out below are indicated in various oral care products at various levels as detailed in the examples below.

TABLE 1
Emulsion/Sialagogue Containing Formulations
suitable for various oral care products of the invention

| | Percentage |
|---|---|
| | (%) by wt. |
| Formula A: | |
| Jambu Oleoresin | 0.1 |
| Poloxamer 407 | 44.3 |
| Polydimethylsiloxane | 17.6 |
| (Dow Corning 1500) | |
| Dicalcium phosphate | 13.3 |
| Carrageenan | 13.3 |
| Flavor | 8.9 |
| Saccharin | 2.2 |
| EDTA | 0.2 |
| Propyl Gallate | 0.1 |
| | 100.0 |
| Formula B: | |
| zingerone | 0.0001 |
| Poloxamer 407 | 62 |
| Hydrogenated Vegetable Oil | 20 |
| Sodium Bicarbonate | 18 |
| | 100 |
| Formula C: | |
| capsaicin | 0.0001 |
| Poloxamer 407 | 91.74 |
| Sodium Fluoride | 1.6 |
| Cetylpyridium Chloride | 0.6 |
| Domiphen Bromide | 0.06 |
| Hydrogenated Vegetable Oil | 5.0 |
| Carrageenan | 1.0 |
| | 100 |
| Formula D: | |
| Eugenol | 0.001 |
| Poloxamer 407 | 87.1 |
| Sorbitol | 10.5 |
| Sodium Fluoride | 1.7 |
| Cetylpyridinium Chloride | 0.63 |
| Domiphen Bromide | 0.07 |
| | 100 |

Examples 1 through 4 include various oral care products with emulsion/sialagogue mixtures of the invention as described in Table 1 above.

### EXAMPLE 1

Formula A was introduced into a standard fluoride toothpaste at 2% by weight.

### EXAMPLE 2

Formula B was introduced into a standard antimicrobial mouth rinse at 1.5% by weight.

### EXAMPLE 3

Formula C was introduced into a breath spray at 1% by weight.

### EXAMPLE 4

Formula D was introduced into a gel at 2% by weight.

What is claimed is:

1. Biofilm-responsive oral care products containing emulsion/sialagogue mixtures, providing the user with increased and prolonged saliva flow.

2. Biofilm-responsive oral care products, comprising an emulsion having a surfactant continuous phase and a coating substance discontinuous phase, containing a sialagogue, providing the user with increased and prolonged saliva flow.

3. Biofilm-responsive oral care products selected from the group consisting of confectioneries, rinses, sprays, gels, pastes and tablets containing emulsion/sialagogue mixtures providing the user with increased and prolonged saliva flow.

4. Biofilm-responsive oral care products according to claim 3, wherein the emulsion comprises a surfactant continuous phase and a coating substance as the discontinuous phase.

5. Biofilm-responsive oral care products according to claim 3, wherein said confectioneries are selected from the group consisting of chewing gums, mints, lozenges, candies and combinations thereof.

6. Biofilm-responsive oral care products according to claim 2, wherein said coating substance is selected from the group consisting of waxes and polydimethyl-siloxanes having a viscosity from between about 1,000 cs and 10 million cs and mixtures thereof.

7. A method for controlling biofilms in the oral cavity comprising periodically using biofilm-responsive oral care products containing emulsion/sialagogue mixtures providing the user with increased and prolonged saliva flow.

8. A method for increasing and prolonging saliva flow comprising periodically introducing into the oral cavity biofilm-responsive oral care products containing emulsion/sialagogue mixtures.

9. A method for manufacturing biofilm-responsive oral care products suitable for providing the end user with increased and prolonged saliva flow, comprising the step of adding emulsion/sialagogue mixtures to said oral care products.

10. A method for treating the oral cavity for increasing saliva production therein, comprising the step of introducing into the oral cavity a biofilm-responsive oral care product containing an emulsion/sialagogue mixture.

11. Biofilm-responsive oral care products containing a substantially water-free emulsion having a surfactant continuous and a coating substance discontinuous phase, where said emulsion contains at least one sialagogue;
wherein said products are suitable for:
(a) controlling, disrupting and physically removing biofilms, and
(b) increasing and prolonging saliva flow.

12. Biofilm-responsive oral care products, according to claim 2, wherein said surfactant continuous phase is selected from the group of surfactants consisting of nonionic, cationic, anionic, amphoteric surfactants and mixtures thereof.

13. Biofilm-responsive oral care products, according to claim 6, wherein said polydimethylsiloxane has a viscosity between about 1000 cs and about 10 million cs.

14. Biofilm-responsive oral care products, according to claim 1, wherein said sialagogue is selected from the group consisting of synthetic and natural plant extracts comprising; N-Isobutyl-2,6,8-decatrienamide, amides of vegetable origin, Heliopsis Longipes, Chiluan Root derivatives, N-isobutylamides from buds of Spilanthes acmella, capsacinia, alkamides of flavoring plants, pepper alkanides, pilocarpine alkanides, ginger alkanides, ginseng parts, Jambo Oleoresin, Zanthoxylom peperitum, sanshool- 1, Sanshool-II, sanshoamide, isopulegole, 3-(1-menthoxy) propan-1,1-diol, p-menthan-3,8-diol, 6-isopropyl-9-methyl-1,4-dioxaspiro-(4,5)-decane-2-methanol, menthyl succinate, menthyl succinate, trimethyl cyclohexanol, N-ethyl-2-isopropyl-5-methylcyclohexane carboxamide, 3 -(1 -menthoxy)-2-methyl-propan- 1,2-diol, menthone glycerin ketal, menthyl lactate, [1′R,2′S,5′R]-2-(5′methyl-2′-(methylethyl) cyclohexyloxy)ethan-1-ol, [1′R,2′S,5′R]-3-(5′-methyl-2′-(methylethyl)cyclohexyloxy)propan-1-ol, [1′R,2′S,5′R]-4-(5′-methyl-2′-(methylethyl)cyclohexyloxy)butan-1-ol, vanillyl ethyl ether, vanillyl propyl ether, vanillin propylene glycol acetal, ethyl vanillin propylene glycol acetal, gingerol, vanillyl butyl ether, 4-(1-menthoxy-methyl)-2-phenyl-1,3-dioxolane, 4-(1-menthoxy-methyl)-2-(3′,4′-dihydrox-phenyl)-1,3-dioxolane, 4-(1-menthoxy-methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′4′-methylenedioxy-phenyl)-1,3-dioxolane, nonyl acid vanillylamide, 4-(1-menthoxy-methyl)-2-(3′-methoxy-4′-hydroxyphenyl)-1,3-dioxolane, ascorbic acid, black pepper, ginger, licorice, pilocarpine, affinin, spilanthol, bethanechol chloride, cayenne pepper, echinacea, verba santa, bay berry, sanguinarine, ginseng, kava, kudzu, capsaicin, zingerone, eugenol, piperine, and mixtures thereof.

15. Biofilm-responsive oral care products, according to claim 2, wherein said sialagogue also imparts anti-inflammatory activity involving inhibiting cytokines, free radicals and prostaglandins.

16. A method for patient self-treatment for removing, disrupting and controlling biofilms and treating residual biofilms, while increasing and prolonging saliva flow, comprising the step of periodically introducing into the oral cavity, oral care products which are:
(a) biofilm-responsive,
(b) comprise a substantially water-free emulsion having a surfactant continuous phase and a polydimethylsiloxane discontinuous phase, and
(c) wherein said emulsion contains at least one sialagogue.

17. A method for patient self-treatment for dry mouth comprising the step of periodically introducing into the oral cavity, oral care products containing a biofilm-responsive, water-free emulsion having a surfactant continuous phase and a polydimethylsiloxane discontinuous phase, wherein said emulsion contains at least one sialagogue.

18. A method for manufacturing biofilm-responsive oral care products coated with a biofilm-responsive coating comprising the step of adding an emulsion/sialagogue mixture to said oral care products.

19. Biofilm-responsive oral care products containing a biofilm-responsive, saliva soluble, water-free emulsion having a surfactant continuous phase and a polydimethylsiloxane discontinuous phase, wherein said emulsion contains at least one sialagogue, wherein said saliva soluble emulsion is selected from the group consisting of high melt viscosity mixtures and emulsions; medium melt viscosity mixtures and emulsions; and low melt viscosity mixtures and emulsions, and combinations thereof.

20. Biofilm-responsive oral care products, according to claim 2 containing liquid surfactants represented by the general formula: 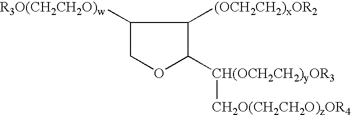
wherein R₁, R₂, R₃, R₄ and H or aliphatic acyl groups having from between about 10 and 30 carbon atoms, and the sum of w, x, y, and z is from between about 20 and about 80.

21. Biofilm-responsive oral care products, according to claim 2, wherein said surfactant continuous phase is selected from the group consisting of:
sodium lauryl sulfate,
sodium lauryl sarcosinate,
polyethylene glycol stearate,
polyethylene glycol monostearate,
coconut monoglyceride sulfonates,
sodium alkyl sulfate,
sodium alkyl sulfoacetates,
block copolymers of polyoxyethylene and polyoxybutylene,
allylpolyglycol ether carboxylates,
polyethylene derivatives of sorbitan esters,
propoxylated cetyl alcohol,
block copolymers comprising a cogeneric mixtures of conjugated polyoxypropylene, and polyoxyethylene compound having as a hydrophobe a polyoxypropylene polymer of at least 1200 molecular weight,
soap powder, and
mixtures thereof.

22. Biofilm-responsive oral care products according to claim 2, wherein said coating substance is selected from the group consisting of polydimethylsiloxanes from between about 1000 cs and about 10 million cs; microcrystalline waxes; paraffin wax; carnauba wax; beeswax; animal fats and animal oils; low melt point, orally suitable polymers; low melt point, orally suitable copolymers and mixtures thereof.
